(19) United States
(12) Patent Application Publication
(10) Pub. No.: US 2008/0220420 A1
(43) Pub. Date: Sep. 11, 2008

(54) Method of Detecting Gene Polymorphism, Method of Diagnosing, Apparatus Therefor, and Test Reagent Kit

(75) Inventors: Yusuke Nakamura, Yokohama-shi (JP); Toshihiro Tanaka, Yokohama-shi (JP); Yozo Ohnishi, Tokyo (JP); Nobuhiro Hanafusa, Kyoto (JP); Koretsugu Ogata, Kyoto (JP); Satoshi Yomota, Kyoto (JP)

Correspondence Address: KRATZ, QUINTOS & HANSON, LLP, 1420 K Street, N.W., Suite 400, WASHINGTON, DC, 20005, US

(73) Assignees: SHIMADZU CORPORATION, Kyoto-shi (JP); RIKEN, Wako-shi (JP)

(21) Appl. No.: 11/667,971

(22) PCT Filed: Nov. 18, 2005
(86) PCT No.: PCT/JP05/21235

§ 371 Date: May 12, 2008

(30) Foreign Application Priority Data
Nov. 19, 2004 (JP) 2004-336607

(51) Int. Cl. C12Q 1/68; C12M 1/00

(52) U.S. Cl. 435/6; 435/287.2

(57) ABSTRACT

The object of the invention is to carry out typing for multiple SNP sites automatically from the stage of sample preparation. A mixture of sample (2) and PCR reaction solution (4) is subjected to PCR reaction according to a given temperature cycle. After the completion of PCR reaction, invader reagent (6) is added thereto. Subsequently, the reaction mixture having the invader reagent (6) added thereto is added to probe fixing part (8) of typing reaction zone to thereby effect reaction therebetween. Invader probes capable of emitting fluorescence in respective correspondence to multiple SNP sites are separately held on individual sites of the probe fixing part (8), so that the reaction mixture reacts with the invader probes and when SNPs corresponding to the invader probes exist, fluorescence is emitted.

### TECHNICAL FIELD

The present invention relates to a method, an apparatus and a reagent for detecting genome DNA polymorphism for plants and animals including humans, particularly SNP (single-nucleotide polymorphism), and a method and apparatus for diagnosing disease morbidity, the relationship between the type and effect or side effect of a drug administered, etc. by using the result of the above detection.

The method and apparatus for detecting gene polymorphism can be utilized in the study of gene analysis and in the clinical field.

### BACKGROUND ART

A method and apparatus for estimating susceptibility to diseases, etc., by using gene polymorphism have been proposed as follows:

For determining whether a patient is susceptible to sepsis and/or rapidly develops sepsis, a nucleic acid sample is collected from the patient, a pattern 2 allelic gene or a marker gene which is in linkage disequilibrium with a pattern 2 allelic gene in the sample is detected, and if a pattern 2 allelic gene or a marker gene in linkage disequilibrium with a pattern 2 allelic gene is detected, the patient is judged to be susceptible to sepsis (see Patent Literature 1).

For diagnosis of one or more single-nucleotide polymorphisms in the human flt-1 gene, a sequence of one or more positions in human nucleic acid, that is, positions 1953, 3453, 3888 (which are respectively in accordance with numbering in EMBL Accession No. X51602), 519, 786, 1422, 1429 (which are respectively in accordance with numbering in EMBL Accession No. D64016), 454 (in accordance with Sequence No. 3) and 696 (in accordance with Sequence No.: 5) is determined, and by referring to the polymorphism in fl1-1 gene, the constitution of the human is determined (JP-A 2001-299366).

Many methods have been reported on typing, that is, discrimination of bases in SNP sites. A typical example of these methods is as follows:

For carrying out typing several hundred thousand SNP sites with a relatively small amount of genome DNA, a plurality of base sequences containing at least one single-nucleotide polymorphism are amplified simultaneously with a genome DNA and pairs of primer, and a plurality of base sequences thus amplified are used to discriminate bases in single-nucleotide polymorphic sites contained in the base sequences by a typing step. For the typing step, an invader method or TaqMan PCR is used (see Patent Literature 3).

For typing of SNPs, however, preparation of genome DNA is essential at a stage leading to the amplification step, which requires much time and high costs.

When attention is focused on PCR for amplifying DNA, a method wherein a sample such as blood is subjected to PCR directly without pre-treating the sample has also been proposed. In this nucleic acid synthesis method of amplifying a desired gene in a gene-containing sample, a gene inclusion body in the gene-containing sample, or the gene-containing sample itself, is added to a gene amplification reaction solution to amplify the desired gene in the gene-containing sample in the reaction solution at pH 8.5 to 9.5 (25° C.) after addition (see Patent Literature 4).

### Patent Literature 1: Japanese Patent Application National Publication (Laid-Open) No. 2002-533096

### Patent Literature 2: JP-A 2001-299366

### Patent Literature 3: JP-A 2002-300894

### Patent Literature 4: Japanese Patent No. 3452717

### Patent Literature 5: Japanese Patent No. 3494509

Non-Patent Literature 1: Hsu T. M., Law S. M, Duan S, Neri B. P., Kwok P. Y., “Genotyping single-nucleotide polymorphisms by the invader assay with dual-color fluorescence polarization detection”, Clin. Chem., 2001 August; 47(8):1373-7

### DISCLOSURE OF THE INVENTION

### Problems to be Solved by the Invention

In the previously constructed typing system, the amount of DNA first collected may be small because a plurality of SNP areas to be subjected to typing are amplified by PCR, but the pretreatment, that is, the extraction of DNA from a biological sample is required prior to amplification with PCR. The pretreatment is time-consuming and troublesome.

On one hand, a method of direct PCR amplification of a biological sample such as blood without extracting nucleic acid from the sample has also been previously established, but when the direct PCR method is combined with the typing method, an automatic system wherein a plurality of SNP sites as the object of typing are simultaneously amplified has still not been established. The object of the present invention is to enable typing for a plurality of objective SNP sites automatically from the stage of sample preparation.

### Means for Solving the Problems

The method of detecting gene polymorphism according to the present invention comprises an amplification step of amplifying genome DNA by allowing a biological sample not subjected to a nucleic acid extraction procedure to act directly on a gene amplification reaction solution containing a plurality of primers binding to a plurality of polymorphic sites by sandwiching each site between primers and a typing step of discriminating bases in a plurality of the polymorphic sites by allowing a typing reagent prepared so as to correspond to a plurality of the polymorphic sites to act on the genome DNA amplified in the amplification step.

The relationship between the polymorphic sites and primers is as follows: For amplifying one polymorphic site, a pair of primers binding to the polymorphic site by sandwiching it between primers is necessary. A plurality of kinds of polymorphic sites occur in a target biological sample, and when polymorphic sites occur in positions separated from one another, twice as many kinds of primers as kinds of polymorphic sites are necessary. However, when two polymorphic sites are close to each other, amplification thereof can be effected by binding the primers to each of the polymorphic sites by sandwiching each site between primers or by binding the primers to both sides of a sequence of the two polymorphic sites with no primer between the polymorphic sites. Accordingly, the types of necessary primers are not always twice as many as kinds of polymorphic sites. In the present invention, “a plurality of primers each binding to polymorphic sites by sandwiching each site between primers” is intended to refer to types of primers necessary for amplifying a plurality of polymorphic sites not only in the case where a pair of primers bind to one polymorphic site by sandwiching it between primers but also in the case where a pair of primers bind to two or more polymorphic sites by sandwiching a series of such polymorphic sites between primers.

The polymorphism includes mutation, deletion, overlap, transfer etc. A typical example is SNP.

The nucleic acid extraction procedure as used herein refers to a series of procedures ranging from decomposition of nucleic acid inclusion bodies (membrane structures containing nucleic acid therein, such as cells, microorganisms, fungi, viruses etc.) to extraction of a nucleic acid from the decomposed nucleic acid inclusion bodies. The decomposition of nucleic acid inclusion body is carried out by using, for example an enzyme, a surfactant, a chaotropic agent etc. Extraction of nucleic acid from the decomposed nucleic acid inclusion body is carried out by using, for example, phenol or phenol/chloroform etc.

Accordingly, the biological sample not subjected to the nucleic acid extraction procedure is a sample not subjected to a series of such procedures and includes a nucleic-acid-body-containing-biological sample, a biological sample of nucleic acid inclusion body in a state decomposed by heat treatment or freezing treatment, and a nucleic acid inclusion body recovered from a living sample. The method of recovering a nucleic acid inclusion body from a living sample includes, for example, methods using centrifugation or ultracentrifugation or with a coprecipitating agent such as ethylene glycol etc. or with an adsorbent carrier, etc.

As used herein, the biological sample refers to an animal or plant tissue, a body fluid, excrements, etc., and the body fluid includes blood and saliva. The genome DNA includes DNA such as human, animal or plant DNA, bacterial or viral DNA, and cDNA synthesized with RNA as a template.

In the amplification step, PCR or the like can be used. In this case, PCR is carried out preferably under the pH condition of 8.5 to 9.5 at 25° C.

The step of amplifying genome DNA from a biological sample not subjected to the nucleic acid extraction procedure described above is described in detail in Patent Literatures 4 and 5.

In the typing step, the invader method or TaqMan PCR can be used.

In the diagnostic method of the present invention, diagnostic values with respect to a specific polymorphism or a combination of polymorphisms are prepared as a database, and based on the result of polymorphism detected by the method of detecting gene polymorphism according to the present invention, diagnostic values are read out from the database. The diagnostic values can include disease morbidity, the relationship between the type and effect or side effect of a drug administered, etc.

One aspect of the apparatus for detecting gene polymorphism according to the present invention is constituted to detect gene polymorphism automatically by having a sample arrangement part for arranging a biological sample not subjected to a nucleic acid extraction procedure, an amplification reagent holding part for holding a gene amplification reaction solution containing a plurality of primers binding to a plurality of polymorphic sites by sandwiching each site between primers, a typing reagent holding part which holds a typing reagent prepared so as to correspond to a plurality of the polymorphic sites, an amplification part to control the temperature of a reaction solution having the biological sample added to the gene amplification reaction solution to amplify genome DNA in the reaction solution, a typing reaction part which has a probe fixing part for holding a probe emitting fluorescence corresponding to each of a plurality of the polymorphic sites and which controls the temperature of a reaction solution of the typing reagent and genome DNA amplified in the amplification part in order to react the reaction solution with each probe, a dispensing device which can transfer to the sample arrangement part, the amplification reagent holding part, the typing reagent holding part, the amplification part and the typing reaction part and which dispenses a sample, an amplification reagent, a typing reagent, and a reaction solution of the sample and these reagents to predetermined positions, a fluorescence detector for detecting fluorescence upon irradiation of each probe fixing part in the typing reaction part with an exciting light, and a control part for controlling the temperature of the amplification part and the typing reaction part, the dispensing operation of the dispensing device and the detection operation of the fluorescence detector.

Each probe fixing part can hold not only one kind of probe but also two or more kinds of probes. When two or more kinds of probes are held on one probe fixing part, the respective probes are arranged to be separated from one another so that two or more kinds of fluorescence generated from the respective probes can be distinguishably detected.

In one example of the typing reaction part, each of the probe fixing parts is provided with a concave part, the upper part of which is opened to feed a reaction solution. In this case, the apparatus for detecting gene polymorphism further has an oil holding part to hold oil for the prevention of evaporation of the reaction solution, and the dispensing device is preferably the one capable of dispensing the oil to the concave portion before or after dispensing the reaction solution to the concave portion.

In another example of the typing reaction part, a flow path through which a reaction solution is fed is arranged in each of the probe fixing parts. The flow path may be provided in each of the probe fixing parts with an inlet for feeding the reaction solution and an outlet for discharge and may be connected to a common inlet for feeding the reaction solution and a common outlet for discharge. In this case, the probe fixing part may be formed as a concave portion in the flow path.

An additional example of the typing reaction part is provided with a flow part having a plurality of the probe fixing parts formed therein.

The sample arrangement part and the amplification part may share a temperature regulation part.

The examination reagent kit of the present invention is a kit wherein an amplification reagent holding part in which a gene amplification reaction solution containing a plurality of primers binding to a plurality of polymorphic sites by sandwiching each site between primers has been held, a typing reagent holding part in which a typing reagent prepared so as to correspond to a plurality of the polymorphic sites has been held, and a plurality of probe fixing parts in which probes emitting fluorescence corresponding to each of a plurality of the polymorphic sites have been separately held, are integrally formed.

In the examination reagent kit, a diluent holding part for holding a diluent diluting a sample may further be integrally formed.

Another aspect of the apparatus for detecting gene polymorphism according to the present invention uses the examination reagent kit of the present invention, and comprises an examination reagent kit-mounting part for mounting the examination reagent kit, an amplification part for controlling the temperature of a reaction solution of the gene amplification reaction solution and a body fluid sample for amplifying genome DNA in the reaction solution in the amplification reagent holding part, a typing reaction part for controlling the temperature of a reaction solution of the typing reagent and the genome DNA amplified in the amplification part in order to react the reaction solution with the probe in the probe fixing part, a liquid transferring device for transferring liquid from the amplification reagent holding part to the typing reagent holding part and for transferring liquid from the typing reagent holding part to the probe fixing part, a fluorescence detector for detecting fluorescence upon irradiation of each of the probe fixing parts with an exciting light, and a control part for controlling the temperature of the amplification part and the typing reaction part, the liquid transferring operation of the liquid transferring device, and the detection operation of the fluorescence detector.

By way of example, the liquid transferring device is a dispensing device equipped with a dispensing nozzle and arranged so as to be movable to a necessary place.

Still another aspect of the apparatus for detecting gene polymorphism according to the present invention uses the examination reagent kit of the present invention wherein each of the holding parts is made of a soft material, and the liquid transferring device is a pressing device for pressing to deform each of the holding parts, thereby transferring the liquid.

The diagnostic device of the present invention comprises the apparatus for detecting gene polymorphism according to the present invention, a database in which diagnostic values such as disease morbidity, the relationship between the type and effect or side effect of a drug administered, etc., with respect to a specific SNP or a combination of SNPs are memorized, and a display unit for displaying diagnostic values by reading out from the database on the basis of the result of SNPs detected by the apparatus for detecting gene polymorphism.

FIG. 1 schematically shows the detection method of the present invention. The present invention is described by reference to the detection method wherein the PCR method is used in the amplification step and the invader method is used in the typing step.

In the PCR step, a PCR reaction solution 4 is added to a biological sample 2 such as blood, or alternatively, the biological sample 2 is added to the PCR reaction solution 4. For example, 1 μL of the sample 2 is collected, and about 10 μL of the PCR reaction solution 4 is added thereto. The PCR reaction solution 4 has been previously prepared, and contains a plurality of primers for SNP sites to be measured, a buffer solution for adjusting pH, 4 kinds of deoxyribonucleotides and other necessary reagents, and is prepared to exhibit pH 8.5 to 9.5 when mixed with the sample 2.

A mixed solution of sample 2 and PCR reaction solution 4 is subjected to PCR according to a predetermined temperature cycle. The PCR temperature cycle includes 3 steps, that is, denaturation, primer adhesion (annealing) and primer extension, and this cycle is repeated whereby DNA is amplified. In one example of the steps, the denaturation step is carried out at 94° C. for 1 minute, the primer adhesion step at 55° C. for 1 minute, and the primer extension at 72° C. for 1 minute. The sample is not subjected to genome extraction procedure, and at high temperatures of PCR temperature cycles, DNA is released from hemocytes and cells, and the reagents necessary for the PCR reaction are brought into contact with DNA, whereby the reaction proceeds.

After the PCR reaction is finished, an invader reagent 6 is added. A fluorescence-emitting FRET probe and cleavase (structure-specific DNA degradative enzyme) are contained in the invader reagent 6. The FRET probe is a fluorescent-labeled oligo having a sequence completely irrelevant to the genome DNA, and, irrespective of the type of SNP, its sequence is common.

Next, the reaction solution to which the invader reagent 6 has been added is reacted by addition to the probe fixing part 8 in the typing reaction part. At each site of the probe fixing parts 8, an invader probe and a reporter probe are individually held correspondingly to each of a plurality of SNP sites, and the reaction solution reacts with the invader probe to emit fluorescence if SNP corresponding to the reporter probe is present.

The invader method is described in detail in paragraphs [0032] to [0034] in Patent Literature 3.

Two reporter probes have been prepared depending on each base of SNP and can judge whether the SNP is a homozygote or heterozygote.

In the PCR method in the amplification step used in the present invention, a plurality of objective SNP sites are simultaneously amplified, while a plurality of genome DNAs containing the SNP sites are amplified by PCR directly from a biological sample not subjected to nucleic acid procedure. Accordingly, a gene amplification reaction solution containing a plurality of primers for the SNP sites is allowed to act directly on a biological sample and subjected to PCR under the condition of pH 8.5 to 9.5 at 25° C.

The PCR reaction solution contains a pH buffer solution, salts such as MgCl₂, KCl etc., primers, deoxyribonucleotides and a thermostable synthase. In addition, substances such as a surfactant and protein can be added as necessary.

The pH buffer solution can use not only a combination of tris(hydroxymethyl) aminomethane and a mineral acid such as hydrochloric acid, nitric acid, sulfuric acid or the like, but also various pH buffer solutions. The buffer solution with adjusted pH is used preferably at a concentration of 10 mM to 100 mM in the PCR reaction solution.

The primer refers to an oligonucleotide acting as the starting point for DNA synthesis by the PCR reaction. The primers may be synthesized or isolated from biological sources.

The synthase is an enzyme for synthesis of DNA by primer addition and includes chemically synthesized synthases. Suitable synthase includes, but is not limited to, E. coli DNA polymerase I, E. coli DNA polymerase Klenow fragment, T4 DNA polymerase, Taq DNA polymerase, T. litoralis DNA polymerase, Tth DNA polymerase, Pfu DNA polymerase, Hot Start Taq polymerase, KOD DNA polymerase, EX Taq DNA polymerase, and a reverse transcriptase. The term “thermostable” refers to the property of a compound which maintains its activity even at high temperatures, preferably at 65 to 95° C.

The invader method used in the typing step is a method of typing SNP site by hybridizing an allele-specific oligo with DNA containing SNP as an object of typing, wherein DNA containing SNP as an object of typing, two kinds of reporter probes specific to the each allele of SNP as an object of typing, one kind of invader probe, and an enzyme having a special endonuclease activity by which a structure of DNA is recognized and cleaved are used (see Patent Literature 3).

### EFFECT OF THE INVENTION

In the method of detecting gene polymorphism according to the present invention, a plurality of objective polymorphic sites are simultaneously amplified from a biological sample not subjected to nucleic acid extraction procedure, thereby typing these polymorphic sites simultaneously, thus enabling the typing of polymorphism to be achieved in a short time in the simple process. The diagnostic method of the present invention can be used in the medical field because diagnostic values are read out from a database on the basis of the typing of polymorphisms.

In the apparatus for detecting gene polymorphism in the first aspect of the invention, the typing of a plurality of objective polymorphisms can be automatically carried out by merely initiating measurement after a biological sample not subjected to nucleic acid extraction procedure is arranged in the sample arrangement part.

In the apparatus for detecting gene polymorphism in the second and third aspects of the invention, the typing of a plurality of objective polymorphisms can be automatically carried out in the simple measurement apparatus by using the examination reagent kit having a gene amplification reaction solution, a typing reagent and a diluent accommodated therein, wherein probe fixing parts are integrally formed.

In the diagnostic apparatus of the present invention, processes ranging from the typing of polymorphisms to the indication of diagnostic values based on the typing can automatically be carried out.

### BEST MODE FOR CARRYING OUT THE INVENTION

FIG. 2(A) schematically shows an apparatus for automatically detecting gene polymorphism in one example.

The numeral 10 denotes a sample table serving as a sample arrangement part and a reagent holding part Blood collection tubes 12 are arranged as sample containers in the sample arrangement part. The sample table is loaded with blood collection tubes 12 with various sizes, for example those of 13 mm in diameter, 16 mm in diameter, etc., further loaded with a universal adaptor which can also be loaded with sample cups and is thus capable coping with various sample containers. Blood is collected in the blood collection tube 12 as a body fluid sample not subjected to genome extraction procedure and is mounted on the sample table 10.

The sample holding part in the sample table 10 is loaded with the PCR reaction solution 14 as an amplification reagent and the invader reaction reagent 15 as a typing reagent.

Hereinafter, the present invention is described in detail by reference to the composition of each reaction solution, but the technical scope of the present invention is not limited to the following examples.

PCR is carried out by using 24 μl of the PCR reaction solution for 1 μl of fresh human blood.

50 mmol each of 40 kinds of primers (20 pairs), 10 U EX-Taq DNA polymerase (manufactured by Takara Shuzo Co., Ltd.), 0.55 μg TaqStart (manufactured by CLONTECH Laboratories), and AmpDirect (manufactured by Shimadzu Corporation) are mixed in this PCR reaction solution. As the primers, for example, SNP ID Nos.:1 to 20 and Sequence Nos.:1 to 40 shown in Table 1 in Patent Literature 3 can be used.

As the invader reagent, an invader assay kit (manufactured by Third Wave Technology) is used. That is, a buffer, a FRET probe, a cleavase and distilled water were prepared by mixing them at the ratio of 3:3:3:50.

The numeral 20 denotes a reaction table, and a PCR area 22 is in the inside of the reaction table 20, and amplification reaction containers 24 are arranged. The PCR area 22 is provided with a temperature control part by which the temperature of the reaction solution becomes a temperature established for PCR amplification reaction. The amplification reaction containers 24 are disposable containers made of resin and are formed to have such a thin wall as to improve heat exchange. The temperature of the PCR area 22 is changed at 3 stages, for example at 94° C., 63° C. and 72° C., and established to repeat this cycle.

On the reaction table 20, the invader reaction area 28 is arranged for a typing reaction in a fashion concentric with the PCR area 22, at the side of the outer circumference of the PCR area 22. Typing reaction containers 30 are arranged in the invader reaction area 28, and minute wells 42 which are as many as or several times as many as SNPs to be detected are formed in the typing reaction containers 30. The capacity of well 42 is for example several 10 nL to several μL. The invader reaction area 28 is provided with a temperature control part independent of that in the PCR area 22, so as to make the invader reaction area 28 different in temperature from the PCR area 22. The temperature of the invader reaction area 28 is set for example at 63° C.

A sectional view of the typing reaction containers 30 is shown in FIG. 2(B), and the invader probe and reporter probe 44 corresponding to SNP have been previously fixed to each well 42. The reaction solution containing the DNA amplified by the PCR reaction, and the invader reaction reagent, are dispensed to each well 42 and react with the invader probe 44. When SNPs corresponding to the invader probes 44 are present in the dispensed reaction solution, fluorescence is emitted by the FRET probe.

As a specific example of the reporter probe and invader probe, it is possible to employ, for example, primary probes 1, 2 and invader probe described in Table 1 in Non-Patent Literature 1.

The invader probe and reporter probe have been fixed to each well in an air-dried state.

In order to measure fluorescence from the typing reaction containers 30 in the bottom side of wells 42, the typing reaction containers 30 are made of a light-permeable resin with a low-spontaneous-fluorescent property (that is, a property of generating little fluorescence from it), for example a material such as polycarbonate.

By reference to FIG. 2(A), a fluorescence detector 50 is arranged for measuring fluorescence from the typing reaction container 30. The fluorescence detector 50 includes a laser diode (LD) or light-emitting diode (LED) 52 as an exciting light source for emitting a laser light at 473 nm, and includes a pair of lenses 54, 56 for applying the laser light after collecting it on the bottom of well 42 of the container 30. The lens 54 is a lens for collecting the laser light from the laser diode 52 to convert it into a parallel light, and the lens 56 is an objective lens for applying the parallel laser light after converging it on the bottom of well 42. The objective lens 56 also acts as a lens for collecting fluorescence generated from well 42. A dichroic mirror 58 is arranged between a pair of lenses 54, 56, and the wavelength characteristic of the dichroic mirror 58 is established so that an exciting light passes therethrough, while fluorescence is reflected. A dichroic mirror 60 is further arranged on the optical path of a reflected light (fluorescence) of the dichroic mirror 58. The wavelength characteristic of dichroic mirror 60 is established so that a light at 525 nm is reflected, while a light at 605 nm passes therethrough. On the optical path of a light reflected by the dichroic mirror 60, a lens 62 and a light detector 64 are arranged so as to detect fluorescence at 525 nm, and on the optical path of a light transmitted through the dichroic mirror 60, a lens 66 and a light detector 68 are arranged so as to detect fluorescence at 605 nm. By detecting two kinds of fluorescence with the two detectors 64, 68, the presence or absence of SNP corresponding to the invader probe fixed in each well and whether the SNP is a homozygote or heterozygote can be determined. As the labeled fluorescent substance, for example, FAM, ROX, VIC, TAMRA etc. can be used.

A dispensing probe 32 having a nozzle 34 is arranged between the sample table 10 and the reaction table 20. The nozzle 34 is transferred between the sample table 10 and the reaction table 20, and carries out the operation of suctioning a sample from the blood collection tube 12 arranged in the sample table 10 and dispensing it into the amplification reaction container 24 in the PCR area in the reaction table 20, the operation of dispensing the PCR reaction solution 14 arranged in the sample table 10 into the amplification reaction container 24, the operation of dispensing the invader reaction reagent 15 arranged in the sample table 10 into the amplification reaction container 24, and the operation of dispensing the reaction solution in the amplification reaction container 24 into well 42 in the typing reaction container 30. For manipulating the dispensing operation with the nozzle 34, a syringe pump 38 and washing water 40 are connected via a switching valve 36 to the nozzle 34. The washing water 40 is used in dispensing a fluid and in washing the nozzle 34.

To prevent the reaction solutions in the amplification reaction containers 24 or wells 42 from being dried during measurement of fluorescence, a mineral oil container 17 is also arranged in the sample table 10, and the mineral oil therein is dispensed via the nozzle 34 into the wells 42, and as shown in number 45, the surfaces of the reaction solutions are covered with the oil, whereby the reaction solutions can be prevented from being evaporated.

FIG. 3 is a plan view showing the layout of the tables 10 and 20 and the dispensing probe 32 in this example, and the probe 32 is rotated horizontally with an axis 32a as the center, and simultaneously displaced also vertically to execute the dispensing operation.

The movement in this example is described.

The dispensing probe 32 dispenses a sample in the blood collection tube 12, for example in an amount of 1 to several μL, into the amplification reaction container 24 in the PCR area, and then the dispensing probe 32 dispenses the PCR reaction solution 14, for example in an amount of 5 to 10 μL into the amplification reaction container 24 having the sample dispensed therein. Alternatively, the PCR reaction solution 14 may be first dispensed, and the sample then dispensed. In the amplification reaction container into which the sample and the PCR reaction solution were dispensed, PCR is carried out by repeating a predetermined temperature cycle for 1 to 1.5 hours for example. The sample and the reaction solution are also dispensed in series into other amplification reaction containers 24 in the PCR area, to repeat PCR.

In the amplification reaction container 24 in which PCR was finished, the invader reaction reagent 15 is added via the dispensing probe 32 to, and mixed in, the amplification reaction container 24. The mixture is dispensed by the dispensing probe 32 into a plurality of wells 42 in the typing reaction container 30 in the invader reaction area 28, and the invader reaction is carried out for several minutes to several hours. During the reaction or after conclusion of the reaction, fluorescence is measured by the fluorescence detector 50. After the reaction solution is dispensed into the typing reaction container 30, mineral oil may be dispensed onto the reaction solution in well 42 to prevent evaporation of the reaction solution.

FIG. 4 shows the layout of tables etc. in the gene polymorphism detector in another example. A preheat area 22a is arranged in the inside of the PCR area. The preheat area 22a includes a temperature regulation part maintained at 94° C., and the amplification reaction containers arranged therein can be kept always at 94° C. For exchanging the PCR reaction container 24 with the typing reaction container 30, a PCR reaction container arrangement part 70 and a typing reaction container arrangement part 71 are arranged, and for exchanging the PCR reaction container 24 with the typing reaction container 30, a container transfer arm 72 is arranged.

In the apparatus for detecting gene polymorphism in this example, the amplification reaction container 24 and the typing reaction container 30 are transferred via the container transfer arm 72 to predetermined positions respectively. The amplification reaction containers 24 are transferred to, and held in, both the PCR area 22 and preheat area 22a respectively. First, the sample in the blood collection tube 24 is dispensed into the amplification reaction container 24 in the preheat area 22a and preheated at 94° C. At the beginning of PCR, the amplification reaction container in the preheat area 22a is transferred by the container transfer arm 72 to the PCR area 22.

Similarly to the movement shown in the examples in FIGS. 2 and 3, the PCR reaction solution is then dispensed onto the sample in the amplification reaction container 24 in the PCR area 22, to carry out PCR. After PCR is finished, the invader reaction reagent is dispensed and then the reaction solution in the amplification reaction container 24 is dispensed to a plurality of wells in the typing reaction containers 30 to carry out the invader reaction, whereby the fluorescence is detected with the fluorescence detector.

In this example, the amplification reaction container 24 and the typing reaction container 30, in which the reaction has been finished, are transferred by the container transfer arm 72 to a disposal part and discarded, and new amplification reaction container 24 and typing reaction container 30 are set in predetermined positions in the reaction table.

FIG. 5 shows an additional example of the apparatus for detecting gene polymorphism. In this example, the sample table is omitted, and the sample arrangement part and PCR area are arranged in the same area and share the temperature regulation part, and the amplification reaction containers 24 also serve as sample containers. The PCR reaction solution container 14, the invader reaction reagent container 15 and the mineral oil container 17 are arranged in positions close to the reaction table 20, and can be dispensed via the dispensing probe 32 to the reaction containers 24 in the reaction table 20. The other constitution is the same as in the example in FIG. 2.

The movement in this example is described.

A sample in the blood collection tube, for example in an amount of 1 to several μL, is collected, and dispensed into an amplification reaction container 24 and arranged in the PCR area. At the beginning of PCR, a PCR reaction solution 14, for example in an amount of 5 to 10 μL, is dispensed via a dispensing probe 32 into the amplification reaction container 24 into which the sample has been dispensed. Alternatively, the PCR reaction solution 14 may first be dispensed and the sample then dispensed. In the amplification reaction container into which the sample and the PCR reaction solution were dispensed, PCR is carried out by repeating a predetermined temperature cycle for 1 to 1.5 hours for example. The PCR reaction solution is dispensed in series into other amplification reaction containers 24 in the PCR area, in each of which the other sample has been dispensed, to repeat PCR.

In the amplification reaction container 24 in which PCR was finished, the invader reaction reagent 15 is added via the dispensing probe 32 to, and mixed in, the amplification reaction container 24. The mixture is dispensed by the dispensing probe 32 into a plurality of wells 42 in the typing reaction container 30 in the invader reaction area 28, and the invader reaction is carried out for several minutes to several hours. During the reaction or after conclusion of the reaction, fluorescence is measured by the fluorescence detector 50.

FIGS. 6 to 9 each show another example of the typing reaction container arranged in the invader reaction area.

In a typing reaction container 30a in FIG. 6, a plurality of flow paths 74 are formed in a base material, and one or more invader probes have been fixed to the flow path 74. When a plurality of probes are fixed to one flow path 74, the probes are fixed after being separated from one another so that the respective fluorescence can be distinguishably detected. The base material is formed from a material such as light-permeable resin with a low-spontaneous-fluorescent property so that fluorescence can be measured in the bottom side of the flow path 74.

The base material forming the flow path 74 consists of two substrates 76a and 76b bonded to each other. A groove for the flow path 74 is formed on the surface of one substrate 76a so that the flow path 74 is positioned in the inside of the substrate, while the other substrate 76b is bonded to the flow path-forming surface. Both ends of the flow path 74 are provided with an inlet 78a and outlet 78b for the reaction solution, each of which penetrates through the substrate 76b and is open to the surface of the substrate.

A typing reaction container 30b in FIG. 7, similar to the typing reaction container 30a in FIG. 6, is provided with a flow path 74 in the inside of the base material, and a part 74a large in area is formed in the flow path 74 in the typing reaction container 30b. The part 74a may be deeper than in the other portion of the flow path. The invader probe is fixed to the part 74a.

When the reaction solution is dispensed into the inlet 78a in the typing reaction containers 30a and 30b in FIGS. 6 and 7, the reaction solution penetrates into the flow path 74 and reacts with the invader probe fixed therein, to emit fluorescence where SNP corresponding to the invader probe is present.

The typing reaction container 30c shown in FIG. 8 is in common with the typing reaction containers in FIGS. 6 and 7 in that the flow path 78 is formed between 2 substrates, but is different in that all flow paths having the respective invader probes fixed therein are connected to the common reaction solution inlet 80a and common reaction solution outlet 80b.

When the reaction solution is dispensed into the common inlet 80a in the typing reaction container 30c in FIG. 8, the reaction solution penetrates into all flow paths and reacts with the invader probe fixed to the inside of each flow path 78, to emit fluorescence where SNP corresponding to the invader probe is present.

In the reaction container 30d shown in FIG. 9, the flow path 82 is formed as a chamber of broad width in the inside of the base material, and the inlet 84a and outlet 84b that are open to the surface of the base material are arranged at both sides thereof. In the chamber 82, several types of invader probes 44 are fixed at positions separated from one another.

When the reaction solution is dispensed into the common inlet 84a in the typing reaction container 30d in FIG. 9, the reaction solution penetrates into the flow path chamber 82 and reacts with each invader probe 44, to emit fluorescence where SNP corresponding to the invader probe is present.

FIGS. 10 to 19 show examination reagent kits in the form of a stick used in the detector in other aspects of the invention. In each of the views, (A) is a perspective view, and (B) is a plan view.

Each examination reagent kit includes 3 holding parts, that is, a diluent holding part 88, a PCR reaction solution holding part 90 and an invader reaction reagent holding part 92, each of which is expanded in one side of the substrate, and a plurality of invader probe fixing parts 94 are arranged on the surface of the substrate.

The diluent, reaction solution and reaction reagent is held on the respective holding parts 88, 90 and 92, and the openings of the holding parts 88, 90 and 92 are sealed with a detachable film or plate so as to prevent leakage of the liquid before use. After the film or plate on the opening of the diluent holding part 88 is removed, the sample blood is dispensed by nozzle 95 into the diluent holding part 88. After the sample is dispensed, the opening of the diluent holding part 88 is closed again with the film or plate, and the examination reagent kit is attached to the detector.

Invader probes different from one another have been fixed to the invader probe fixing parts 94, and the material of the substrate at least at a part where the invader probe fixing part 94 is arranged is made of a low-spontaneous-fluorescent, light-permeable resin or the like so that emitted fluorescence can be detected on the back surface.

In the examination reagent kit in FIG. 10, the invader probe fixing parts 94 are arranged to be separated from one another and exposed to the surface of the substrate.

Transfer of the liquid from the holding parts 88, 90 and 92 in this examination reagent kit is carried out by using a dispensing nozzle. Accordingly, the film or plate with which the openings of the holding parts 88, 90 and 92 have been sealed is preferably a film or plate into which the dispensing nozzle can easily penetrate.

After a sample, such as blood, is dispensed by the dispensing nozzle into the diluent holding part 88, this reagent kit is attached to an apparatus for detecting gene polymorphism (FIG. 20) described later. In this apparatus for detecting gene polymorphism, the sample in the diluent holding part 88 in this reagent kit is transferred via the dispensing nozzle to the PCR reaction solution holding part 90, and in the apparatus for detecting gene polymorphism, PCR is carried out in a predetermined temperature cycle. After PCR is finished, the reaction solution in the PCR reaction solution holding part 90 is transferred via the dispensing nozzle into the invader reaction reagent holding part 92 and mixed with the invader reaction reagent. Thereafter, the reaction solution in the invader reaction reagent holding part 92 is dispensed via the dispensing nozzle onto each invader probe fixing part 94. In each invader probe fixing part 94, fluorescence by the invader reaction is generated where the corresponding SNP is present in the sample, and the fluorescence is detected by the fluorescence detector in the apparatus for detecting gene polymorphism.

The examination reagent kit shown in FIG. 11 is the same as in FIG. 10 except for the structure of the invader probe fixing part 94a. The structure of the invader probe fixing part 94a is the same as in the typing reaction container 30a in FIG. 6.

The examination reagent kit shown in FIG. 12 is also the same as in FIG. 10 except for the structure of the invader probe fixing part. In this examination reagent kit, the invader probe fixing part 94b is in the form of flow paths, and the invader probes are fixed in the flow paths. A plurality of flow paths in which the invader probes have been fixed are connected to the common inlet 96a and outlet 96b. Only the inlet 96a and outlet 96b are opened, and the flow paths are formed in the substrate.

In this examination reagent kit, dispensing of the reaction solution into the invader probe fixing part 94b can be accomplished by dispensing it into the inlet 96a only once. The reaction solution dispensed into the inlet 96a penetrates into the flow paths and reacts with the invader probes fixed in the flow paths, and fluorescence by the invader reaction is generated where the corresponding SNP is present in the sample, and the fluorescence is detected by the fluorescence detector in the apparatus for detecting gene polymorphism.

In the examination reagent kit shown in FIG. 13, a plurality of invader probes are separated from one another and fixed to a broad flow path as a chamber shown by symbol 94c. The chamber 94c is formed in the inside of the substrate, and the inlet 96a and outlet 96b are opened. In this case too, dispensing of the reaction solution into the invader probe fixing part 94c can be accomplished by dispensing it into the inlet 96a only once. The reaction solution dispensed into the inlet 96a penetrates into chamber 94c and reacts with the invader probes fixed in the chamber 94c, and fluorescence by the invader reaction is generated where the corresponding SNP is present in the sample, and the fluorescence is detected by the fluorescence detector in the apparatus for detecting gene polymorphism.

In the examination reagent kit shown in FIG. 14, different invader probes are fixed to a plurality of positions of an invader probe fixing part 98 made of a low-spontaneous-fluorescent material such as filter paper and attached to the substrate. The invader probe fixing part 98 is exposed to the surface of the substrate.

In this examination reagent kit, the reaction solution may be dispensed through a nozzle into one end of the invader probe fixing part 98, and the reaction solution is diffused through the material of the invader probe fixing part 98 thereby reacting with the probes fixed to respective positions.

The examination reagent kit shown in FIG. 15, similar to the examination reagent kit shown in FIG. 14, is provided with an invader probe fixing part 96b, which is made of a low-spontaneous-fluorescent material such as a filter paper, having different invader probes fixed to a plurality of positions thereof. In this examination reagent kit, however, the invader probe fixing part 96b is retained by being sandwiched between transparent films or transparent plates, and for dispensing the reaction solution into the invader probe fixing part 98, an inlet 100 communicating with the invader probe fixing part 96b is opened. The reaction solution dispensed into the inlet 100 flows into the invader probe fixing part 96b and diffuses thereby reacting with the probes fixed to the respective positions of the invader probe fixing part 96b.

In the examination reagent kits shown in FIGS. 16 to 19, the holding parts 88, 90 and 92 are made of a soft material, and the holding parts 88, 90 and 92 communicate with one another via the grooves 108 and 110 on the surface of the substrate. Before use, the holding parts 88, 90 and 92 are sealed with a seal or plate so that the holding parts 88, 90 and 92 are mutually isolated. A groove 104 is also formed on the surface of the substrate between the invader probe fixing parts 94b, 94c, 96b and 98 and the invader reaction reagent holding part 92.

Before use, the seal or plate is removed, and the sample is dispensed into the diluent holding part 88. Thereafter, when the examination reagent kit is attached to the apparatus for detecting gene polymorphism, the liquid can flow through the grooves 108, 110 between the holding parts 88, 90 and 92 and pass through the groove 104, whereby the fluid can flow from the invader reaction reagent holding part 92 to the invader probe fixing parts 94b, 94c, 96b and 98.

Sending of liquid between the holding parts 88, 90 and 92 in the apparatus for detecting gene polymorphism and sending of liquid from the invader reaction reagent holding part 92 to the invader probe fixing parts 94b, 94c, 96b and 98 are carried out by squashing the holding parts 88, 90 and 92 in this order by mechanical pressing. That is, when the diluent holding part 88 is squashed, the liquid in the diluent holding part 88 passes through the groove 108 and transfers to the PCR reaction holding part 90. Then, when the PCR reaction solution holding part 90 is squashed, the liquid in the PCR reaction solution holding part 90 passes through the groove 110 and transfers to the invader reagent holding part 92. When the invader reaction reagent holding part 92 is further squashed, the liquid in the invader reaction reagent holding part 92 passes through the groove 104 and transfers to the invader probe fixing parts 94b, 94c, 96b and 98, to cause the invader reaction.

The examination reagent kit shown in FIG. 16 is provided with an invader probe fixing part 94b having the same flow path shape as in FIG. 12. The outlet 106 is arranged at the top of the invader probe fixing part 94b, that is, at the end of the side opposite to the holding parts 88, 90 and 92, the sealing seal or plate is removed to open the opening before use, the liquid is sent from the invader reaction reagent holding part 92 and passes through the invader probe fixing part 94b, and an excess of the liquid is discharged from the outlet 106.

The examination reagent kit shown in FIG. 17 is provided with an invader probe fixing part 94c of the same chamber type as in FIG. 13. This examination reagent kit is also provided with an outlet 106 at the top of the invader probe fixing part 94c, that is, at the end of the side opposite to the holding parts 88, 90 and 92, the sealing seal or plate is removed to open the outlet before use, the liquid is sent from the invader reaction reagent holding part 92 and passes through the invader probe fixing part 94c, and an excess of the liquid is discharged from the outlet 106.

The examination reagent kit shown in FIG. 18 is provided with an invader probe fixing part 98, which is made of a low-spontaneous-fluorescent material such as filter paper, having different invader probes fixed to a plurality of positions thereof similar to that in FIG. 14. The invader probe fixing part 98 is exposed to the surface of the substrate.

The examination reagent kit shown in FIG. 19 is provided with an invader probe fixing part 98, which is made of a low-spontaneous-fluorescent material such as filter paper, having different invader probes fixed to a plurality of positions thereof similar to that in FIG. 15. The invader probe fixing part 98 is retained by being sandwiched between transparent films or transparent plates.

Because the invader probe fixing part 98 in the examination reagent kits in FIGS. 18 and 19 is provided with a material such as filter paper having high hygroscopic power, the material can absorb the liquid sent from the invader reaction reagent holding part 92.

FIG. 20 schematically shows an example of the simplified apparatus for detecting gene polymorphism, and this apparatus for detecting gene polymorphism detects SNPs by using the examination reagent kit 122 shown in FIGS. 10 to 19.

The gene polymorphism detector 120 is provided with a mounting part for mounting a plurality of examination reagent kits 122, in which each of the examination reagent kits 122 is mounted in such a state that a sample has been dispensed into the diluent holding part. The detector 120 is also provided with a movable nozzle 124 for sending the liquid to the examination reagent kits 122 mounted in the mounting part.

Although not shown in the figure, the detector 120 is also provided with an amplification part controlling the temperature of reaction solutions, each consisting of the PCR reaction solution and a biological sample in the PCR reaction solution holding part 90 in the examination reagent kit 122, to amplify genome DNAs in the reaction solutions, and is also provided with a typing reaction part controlling the temperature of reaction solutions, each consisting of a typing reagent and genome DNA amplified in the amplification part, to react the reaction solutions with the probes in the probe fixing parts 94, 94a, 94c, 96b and 98.

The numeral 126 denotes a photometric part as a fluorescence detector and is arranged to detect fluorescence emitted from the invader probe fixing parts in a plurality of the examination reagent kits 122 by moving among the examination reagent kits 122. The typing result judged from the detected fluorescence is displayed on display 128.

The above example is an apparatus for detecting gene polymorphism, which can also be a diagnostic apparatus for disease morbidity, the type and effect or side effect of a drug administered and so on. In this case, the apparatus for detecting gene polymorphism is provided with a database in which diagnostic values such as disease morbidity and the type and effect or side effect of a drug administered with respect to a specific SNP or a combination of SNPs are memorized, or the apparatus is connected to such database in the outside. When the apparatus is connected to the database in the outside, the apparatus can be connected to the database via a special line or a general-purpose communication line. The apparatus for detecting gene polymorphism according to the present invention, when used as a diagnostic apparatus, detects SNP results on the basis of which the diagnostic values are read out from the database and displayed on the display unit.

### INDUSTRIAL APPLICABILITY

The present invention can be used in detecting genome DNA polymorphism for plants and animals including humans, particularly SNP (single-nucleotide polymorphism) and can further be utilized, not only in diagnosing disease morbidity, the relationship between the type and effect or side effect of a drug administered and so on by using the results of the above detection, but also in judgment of the variety of animal, or plant, diagnosis of injections (judgment of the type of invader) etc.

### BRIEF DESCRIPTION OF DRAWINGS

FIG. 1 is a flow chart schematically showing the detection method of the present invention.

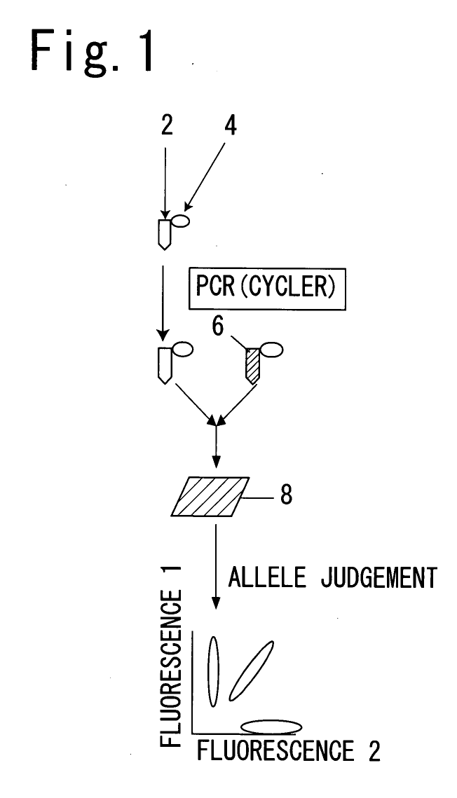

FIG. 2(A) is a perspective view of the main part schematically showing one example of the apparatus for detecting gene polymorphism, and FIG. 2(B) is a partial sectional view of a typing reaction container used therein.

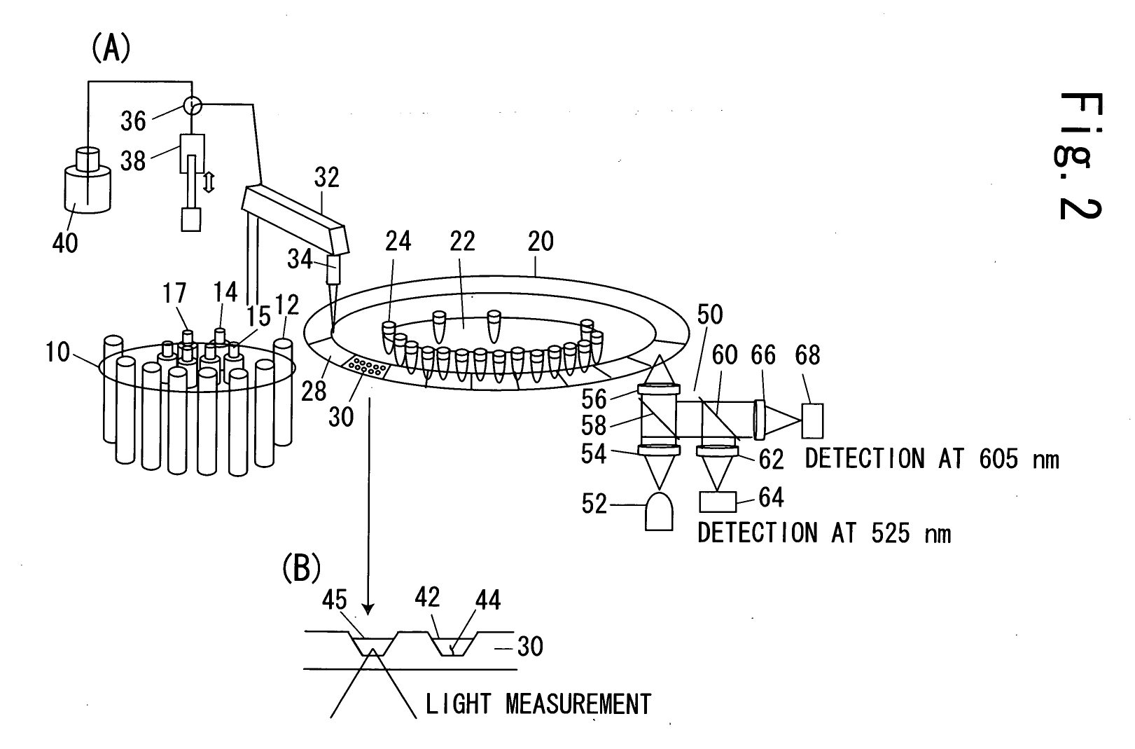

FIG. 3 is a plan view showing the layout of a table and a dispensing probe in the same example.

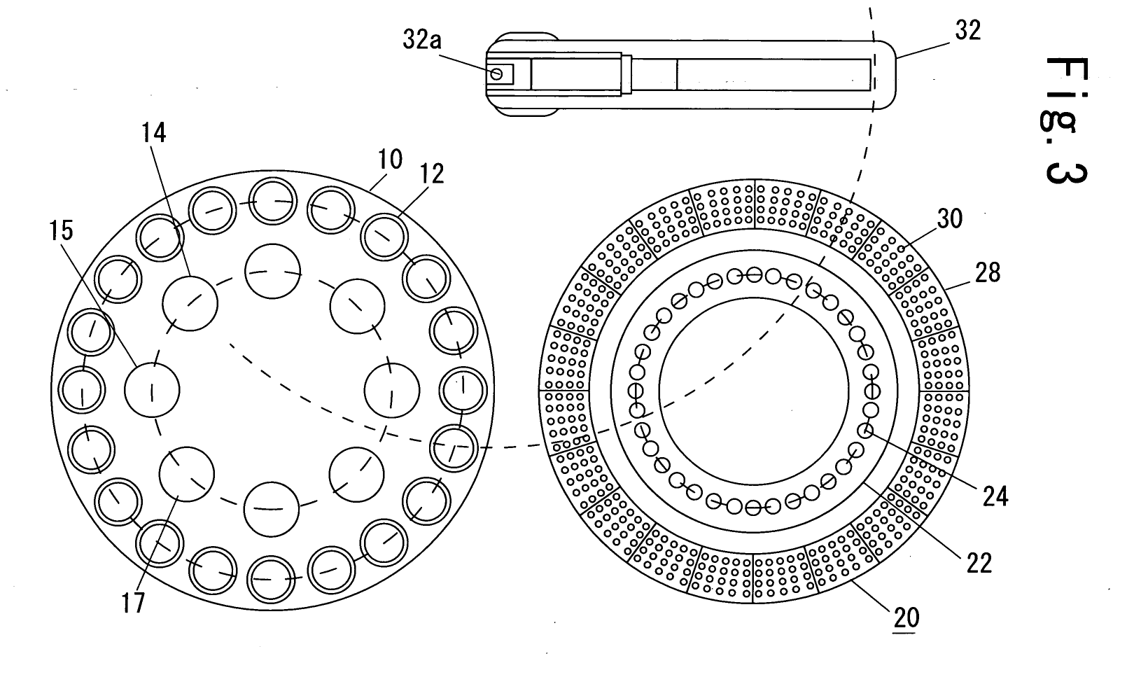

FIG. 4 is a plan view showing the layout of a table and a dispensing probe in another example of the apparatus for detecting gene polymorphism.

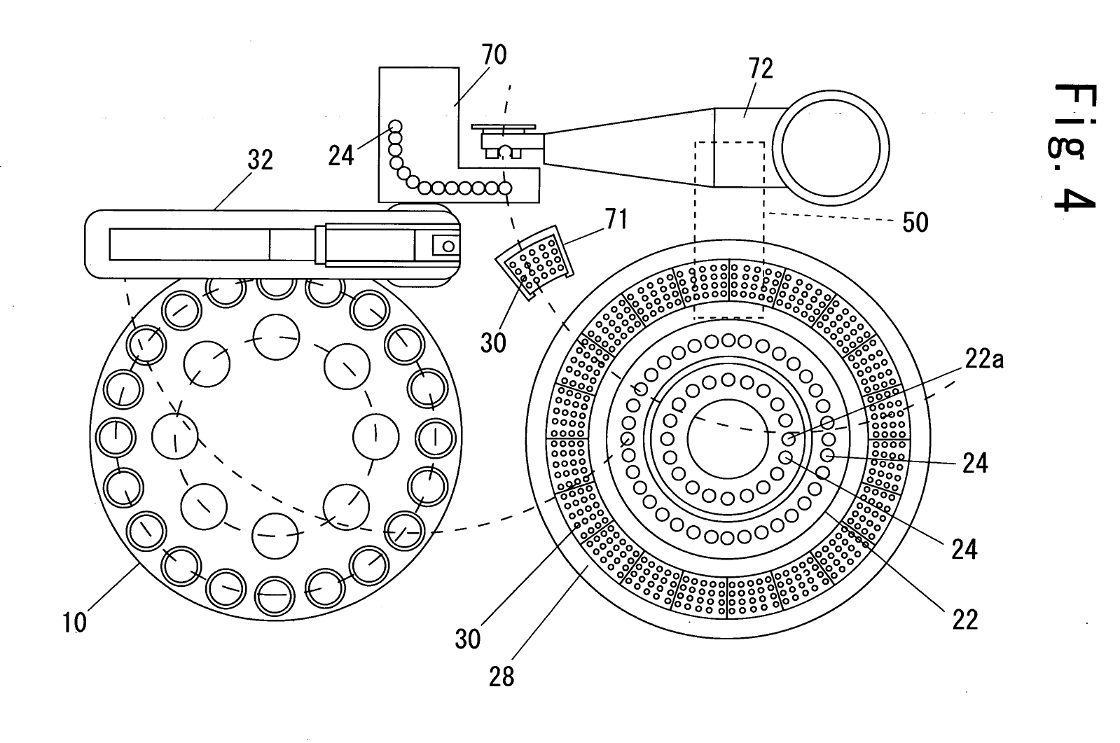

FIG. 5 is a perspective view of the main part schematically showing an additional example of the apparatus for detecting gene polymorphism.

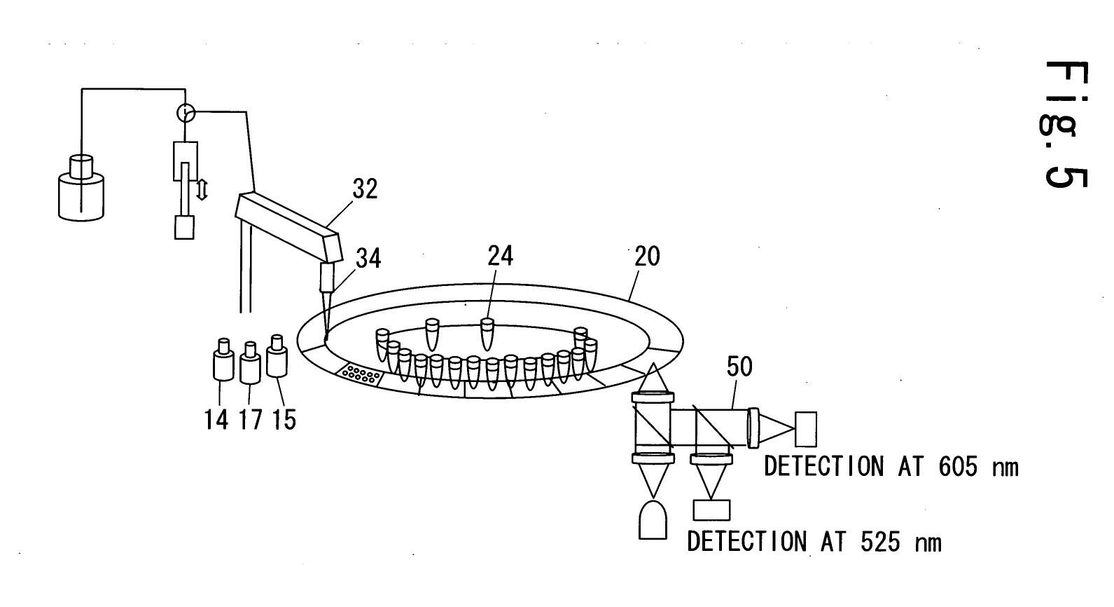

FIGS. 6(A) and 6(B) are views showing another example of the typing reaction container arranged in the invader reaction area, wherein FIG. 6(A) is a plan view and FIG. 6(B) is a sectional view in the position of X-X line in FIG. 6(A).

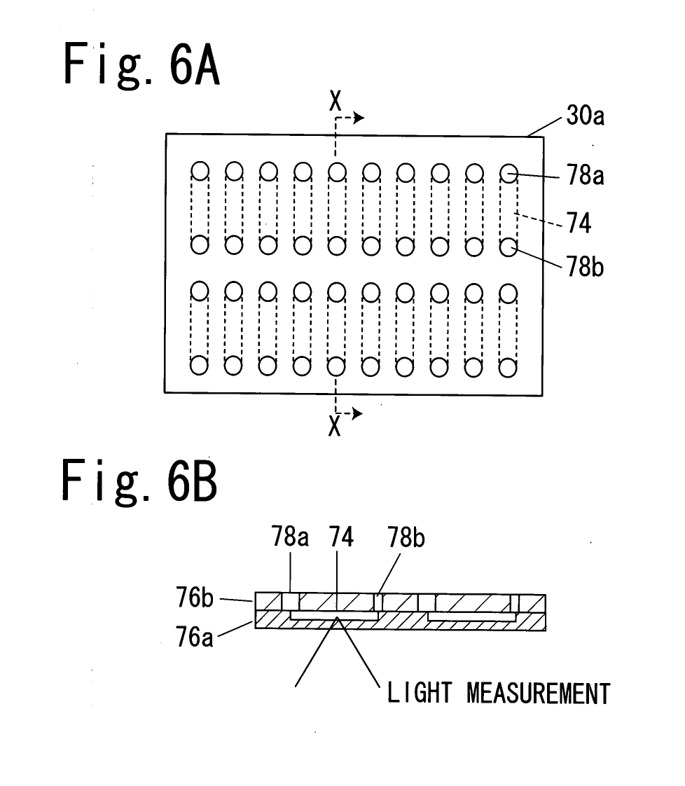

FIG. 7 is a plan view showing an additional example of the typing reaction container arranged in the invader reaction area.

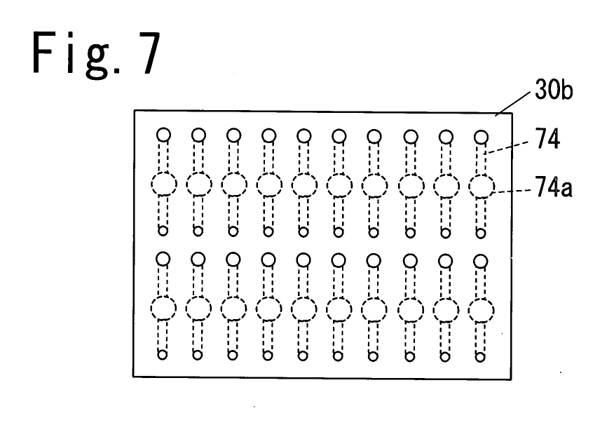

FIGS. 8(A) and 8(B) are views showing an additional example of the typing reaction container arranged in the invader reaction area, wherein FIG. 8(A) is a plan view and FIG. 8(B) is a sectional view along one flow path.

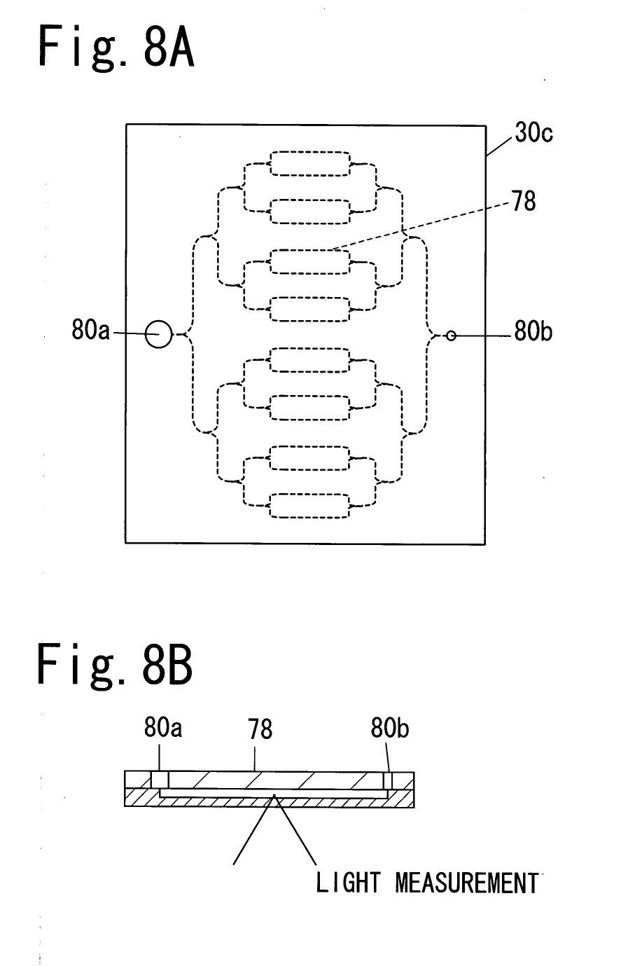

FIGS. 9(A) and 9(B) are views showing an additional example of the typing reaction container arranged in the invader reaction area, wherein FIG. 9(A) is a plan view and FIG. 9(B) is a sectional view in the position of Y-Y line in FIG. 9(A).

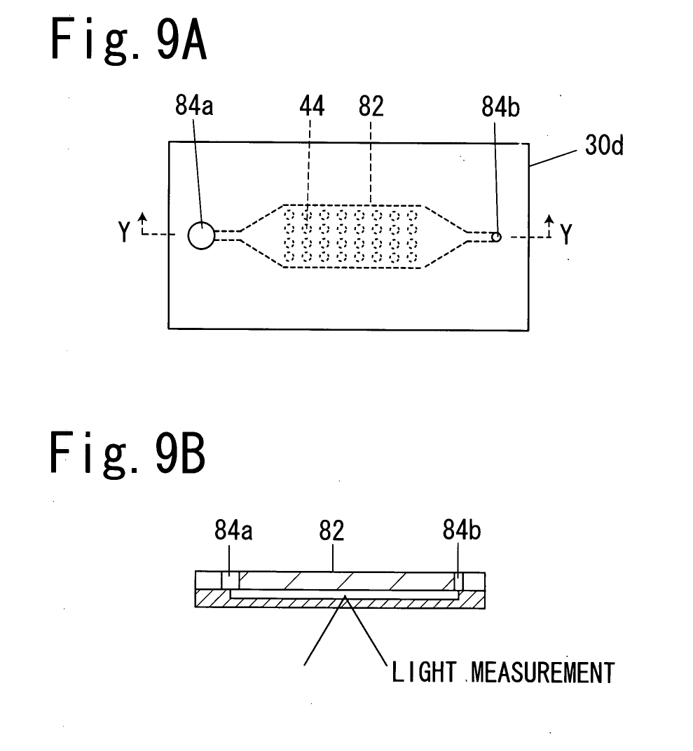

FIGS. 10(A) and 10(B) are views showing one example of the examination reagent kit in the form of a stick, wherein FIG. 10(A) is a perspective view, and FIG. 10(B) is a front view showing the examination reagent kit together with an objective lens of a fluorescence detector.

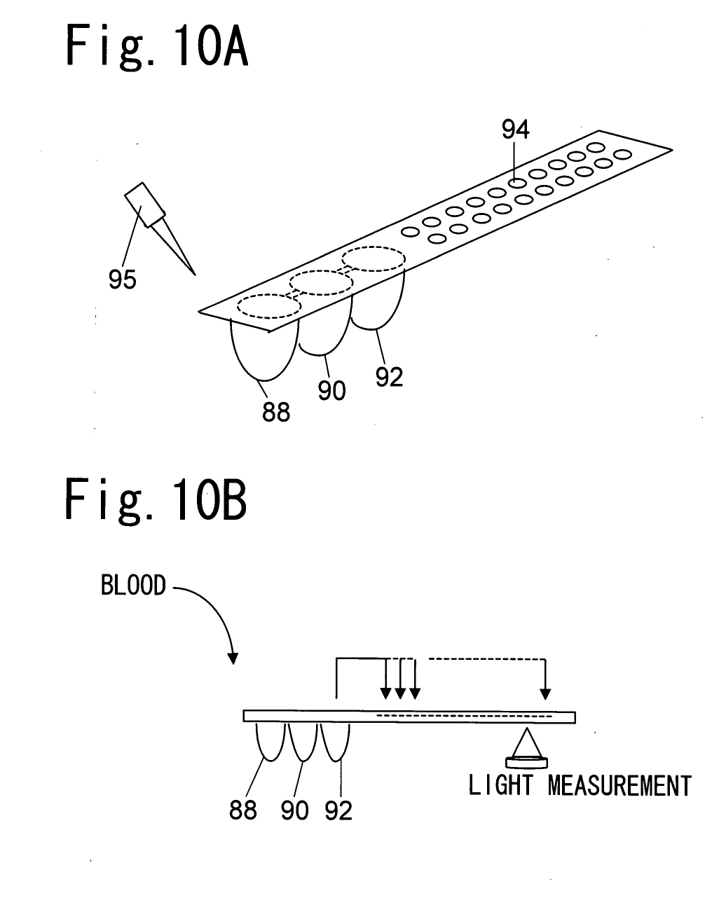

FIGS. 11(A) and 11(B) are views showing another example of the examination reagent kit in the form of a stick, wherein FIG. 11(A) is a perspective view, and FIG. 11(B) is a front view showing the examination reagent kit together with an objective lens of a fluorescence detector.

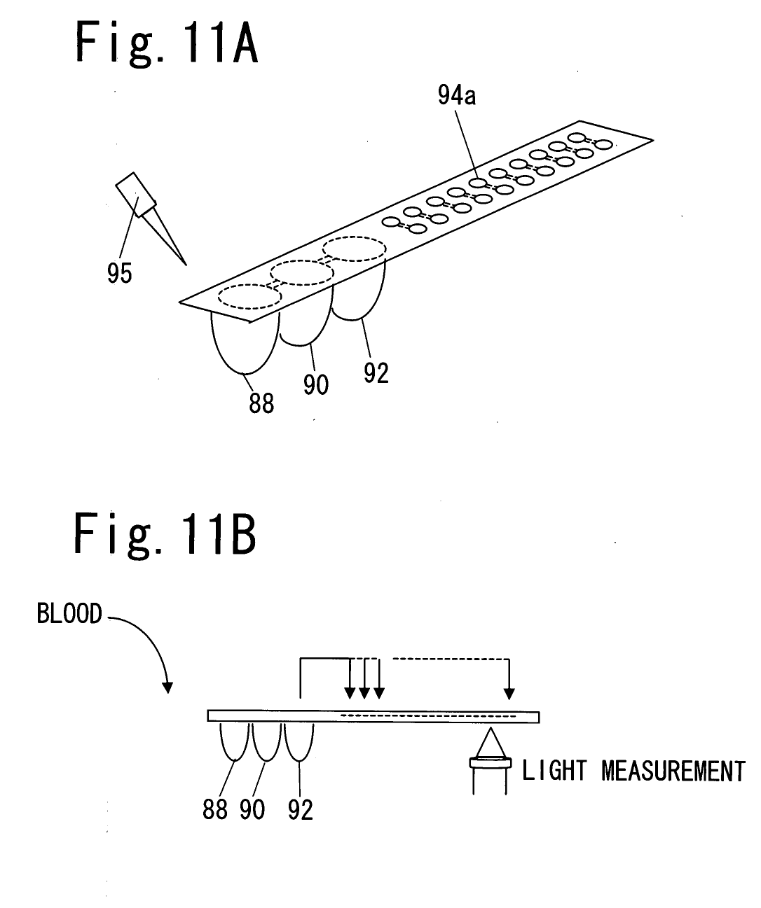

FIGS. 12(A) and 12(B) are views showing an additional example of the examination reagent kit in the form of a stick, wherein FIG. 12(A) is a perspective view, and FIG. 12(B) is a front view showing the examination reagent kit together with an objective lens of a fluorescence detector.

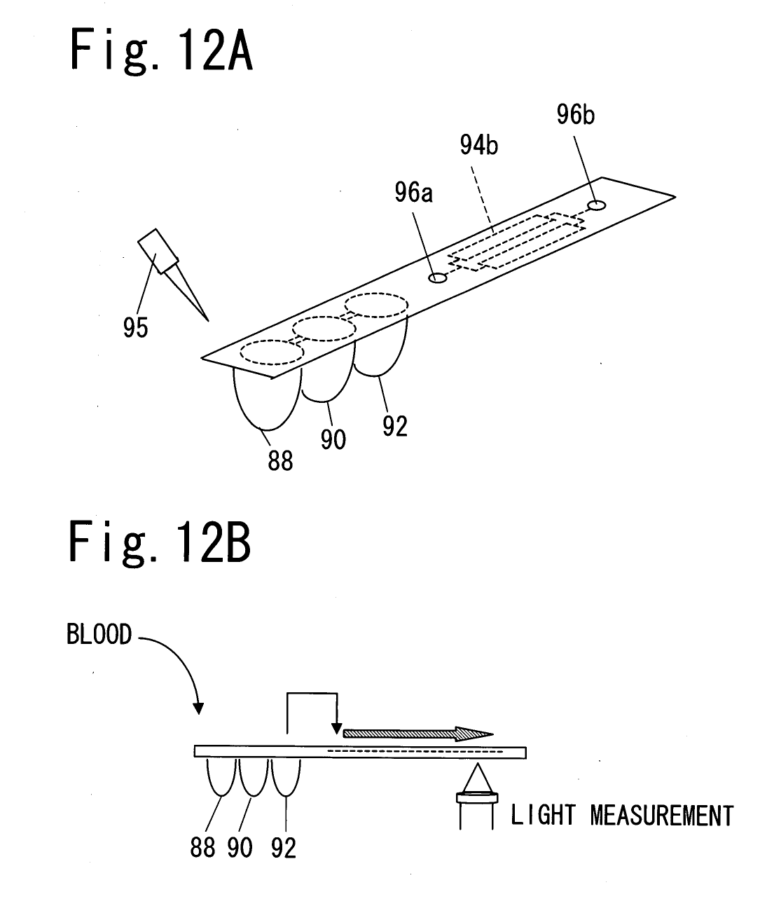

FIGS. 13(A) and 13(B) are views showing an additional example of the examination reagent kit in the form of a stick, wherein FIG. 13(A) is a perspective view, and FIG. 13(B) is a front view showing the examination reagent kit together with an objective lens of a fluorescence detector.

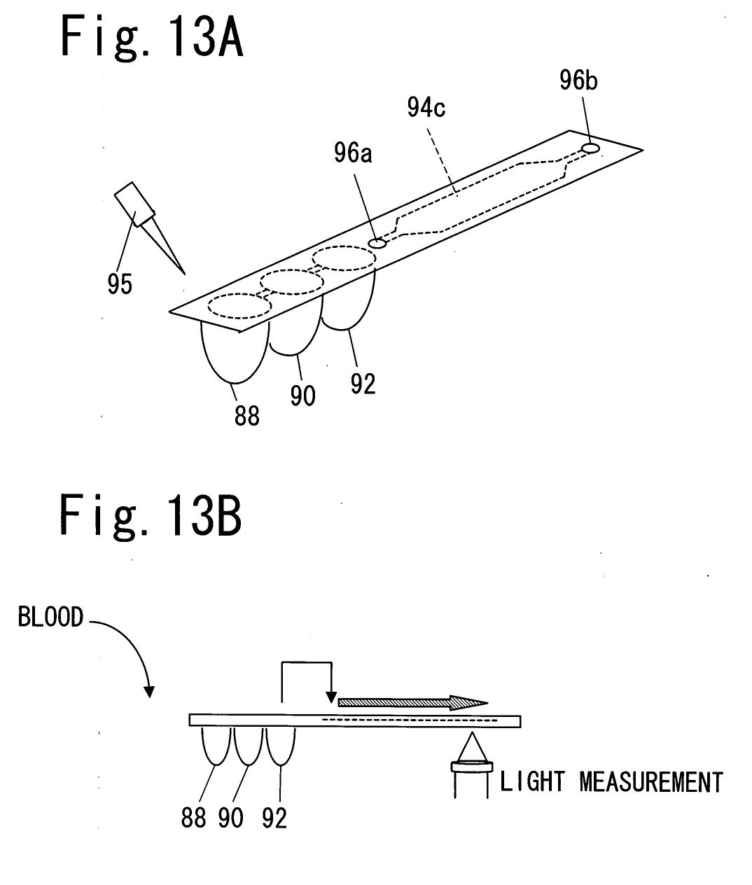

FIGS. 14(A) and 14(B) are views showing an additional example of the examination reagent kit in the form of a stick, wherein FIG. 14(A) is a perspective view, and FIG. 14(B) is a front view showing the examination reagent kit together with an objective lens of a fluorescence detector.

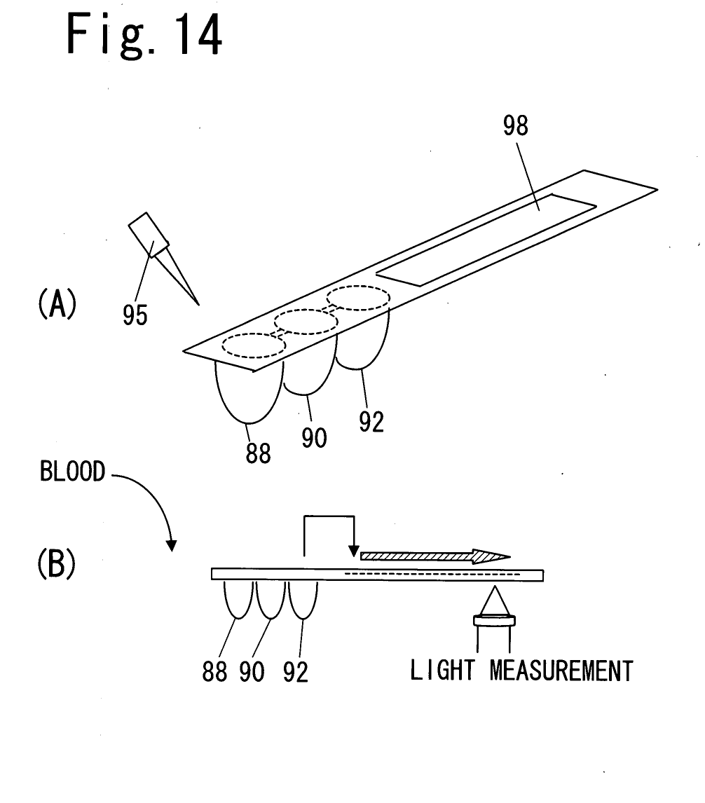

FIGS. 15(A) and 15(B) are views showing an additional example of the examination reagent kit in the form of a stick, wherein FIG. 15(A) is a perspective view, and FIG. 15(B) is a front view showing the examination reagent kit together with an objective lens of a fluorescence detector.

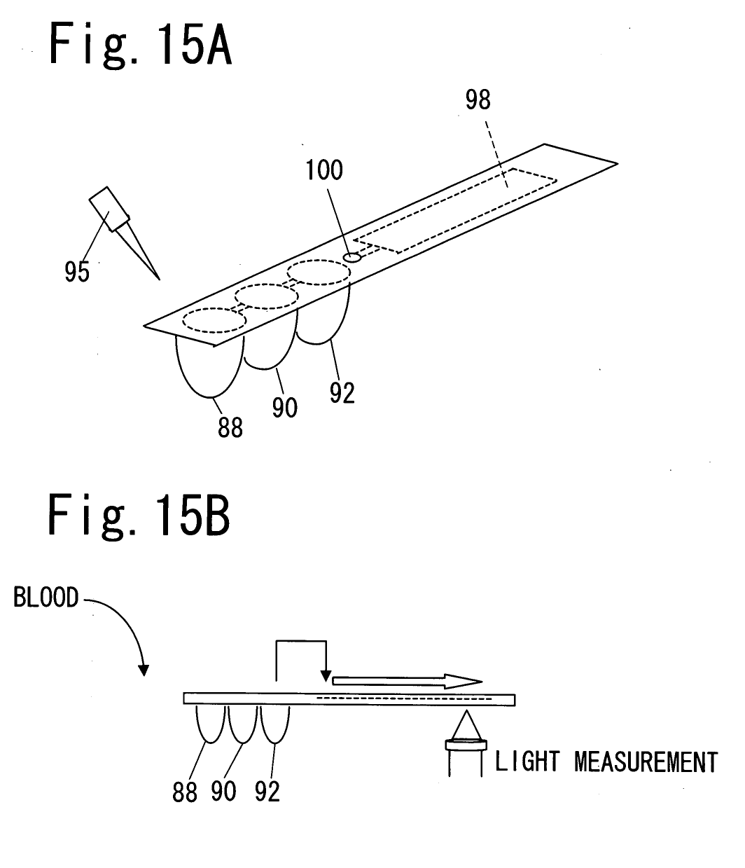

FIGS. 16(A) and 16(B) are views showing an additional example of the examination reagent kit in the form of a stick, wherein FIG. 16(A) is a perspective view, and FIG. 16(B) is a front view showing the examination reagent kit together with an objective lens of a fluorescence detector.

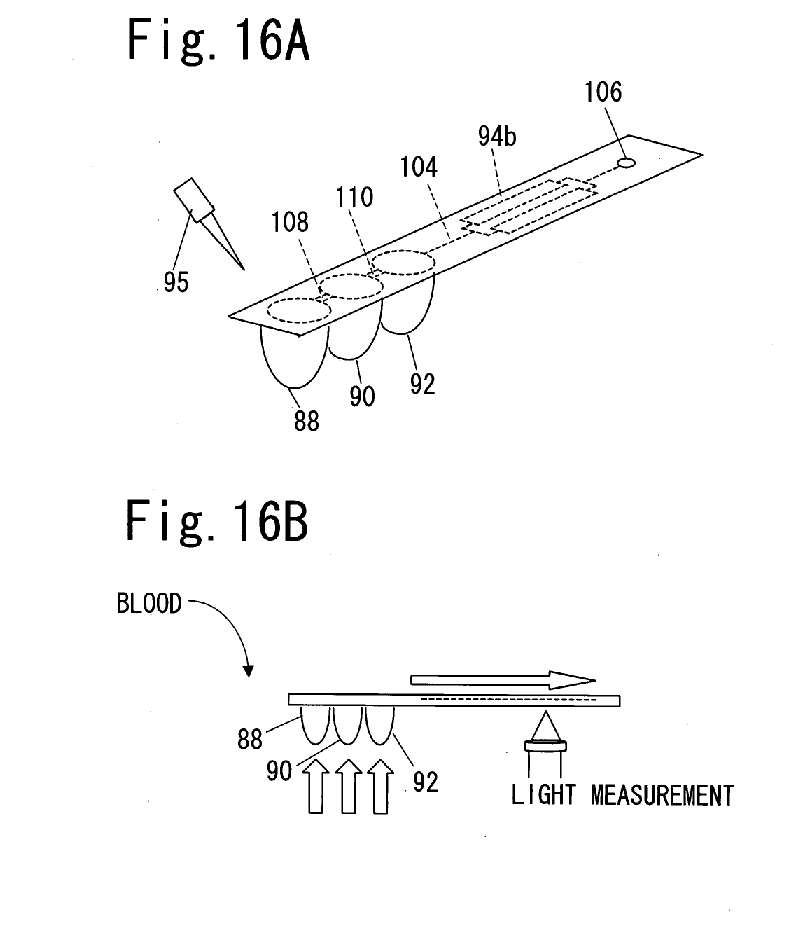

FIGS. 17(A) and 17(B) are views showing an additional example of the examination reagent kit in the form of a stick, wherein FIG. 17(A) is a perspective view, and FIG. 17(B) is a front view showing the examination reagent kit together with an objective lens of a fluorescence detector.

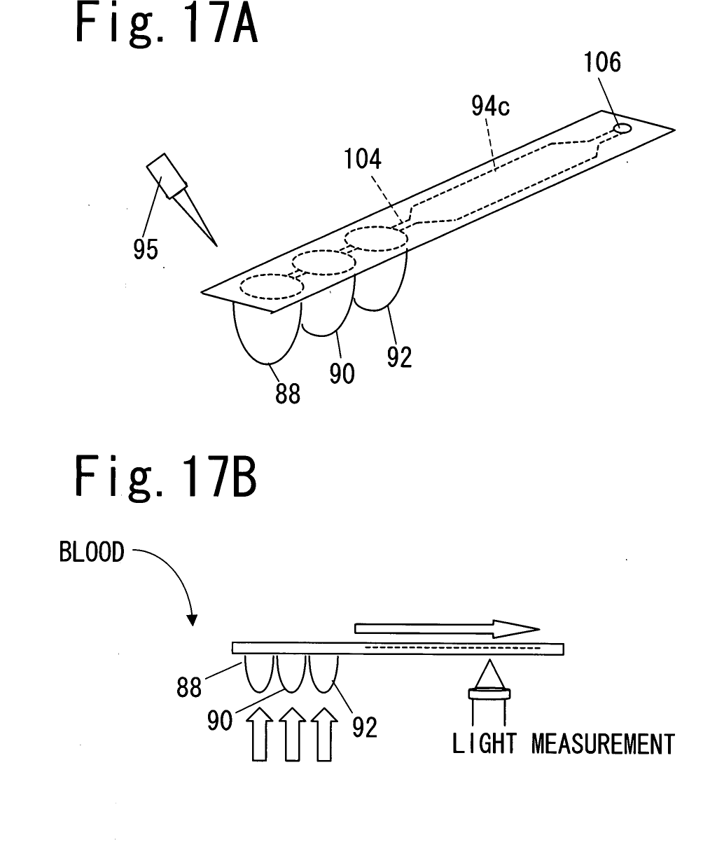

FIGS. 18(A) and 18(B) are views showing an additional example of the examination reagent kit in the form of a stick, wherein FIG. 18(A) is a perspective view, and FIG. 18(B) is a front view showing the examination reagent kit together with an objective lens of a fluorescence detector.

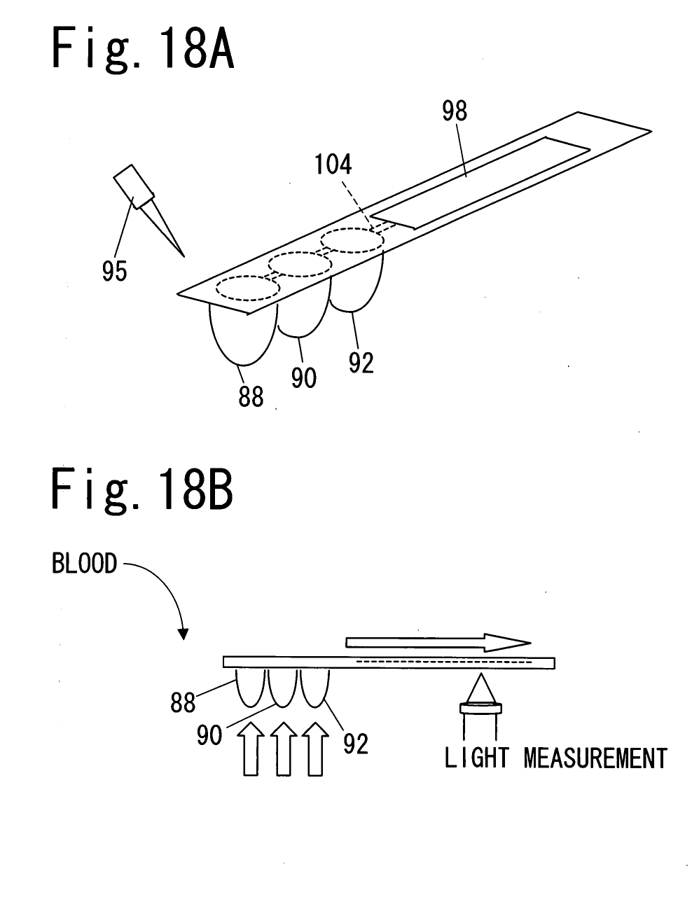

FIGS. 19(A) and 19(B) are views showing an additional example of the examination reagent kit in the form of a stick, wherein FIG. 19(A) is a perspective view, and FIG. 19(B) is a front view showing the examination reagent kit together with an objective lens of a fluorescence detector.

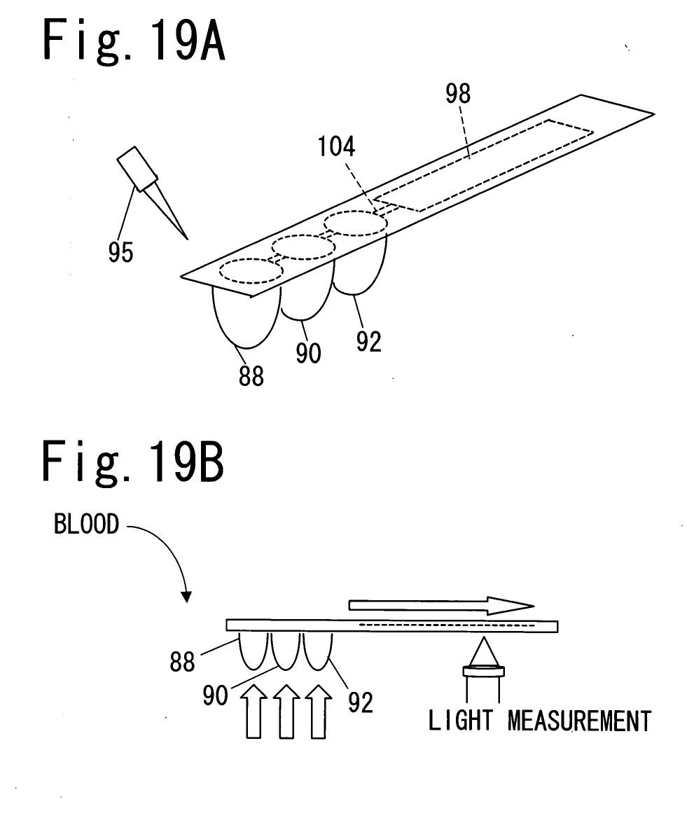

FIG. 20 is a perspective view of the main part schematically showing one example of the simplified apparatus for automatically detecting gene polymorphism.

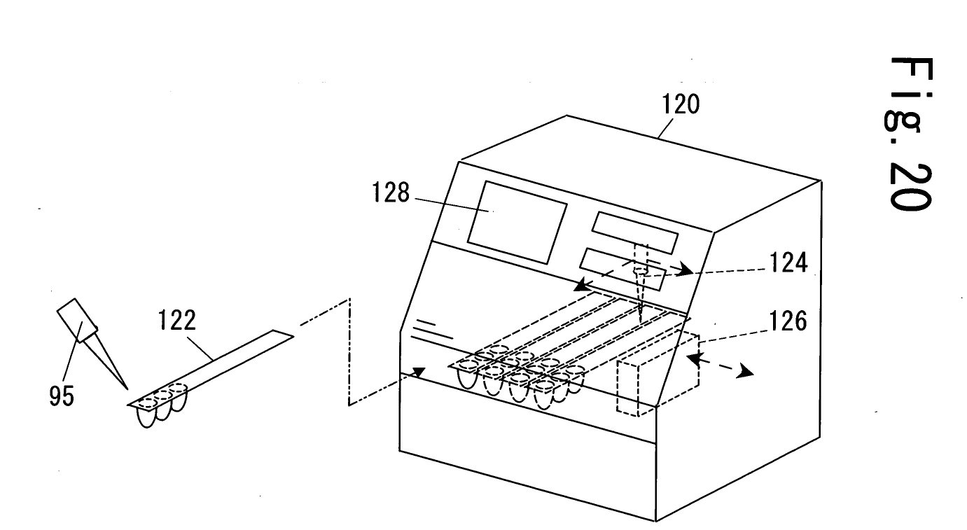

### EXPLANATION OF SYMBOLS

2: sample
4: PCR reaction solution
6: invader reagent
8: probe fixing part
10: sample table
12: blood collection tube
14: PCR reaction solution container
15: invader reaction sample container
17: mineral oil container
20: reaction table
22: PCR area
22a: preheat area
24: amplification reaction container
28: invader reaction area
30, 30a, 30b, 30c, 30d: typing reaction container
34: nozzle
40: washing water
42: well
44: invader probe
45: mineral oil
50: fluorescence detector
70: PCR reaction container arrangement part
71: typing reaction container arrangement part
72: container transfer arm
74, 78: flow path
82: chamber
88: diluent holding part
90: PCR reaction solution holding part
92: invader reaction reagent holding part
94, 94a, 94b, 94c, 96b, 98: invader probe fixing parts
122: examination reagent kit
120: apparatus for detecting gene polymorphism
124: nozzle 124
126: photometric part
128: display

1. A method of detecting gene polymorphism, comprising:
an amplification step of amplifying genome DNA by allowing a biological sample not subjected to a nucleic acid extraction procedure to act directly to a gene amplification reaction solution containing a plurality of primers binding to a plurality of polymorphic sites by sandwiching each site between primers, and
a typing step of discriminating bases in a plurality of the polymorphic sites by allowing a typing reagent prepared so as to correspond to a plurality of the polymorphic sites to act to the genome DNA amplified in the amplification step.

2. The method of detecting gene polymorphism according to claim 1, wherein the objective polymorphism is a single-nucleotide polymorphism.

3. The method of detecting gene polymorphism according to claim 1, wherein the amplification step makes use of a PCR method.

4. The method of detecting gene polymorphism according to claim 1, wherein the typing step makes use of an invader method or a TaqMan PCR method.

5. A diagnostic method, comprising:
preparing, as database, diagnostic values with respect to a specific polymorphism or a combination of polymorphisms, and
reading out diagnostic values from the database on the basis of polymorphism results detected by the method of detecting gene polymorphism according to claim 1.

6. An apparatus for detecting gene polymorphism, comprising:
a sample arrangement part for arranging a biological sample not subjected to a nucleic acid extraction procedure,
an amplification reagent holding part for holding a gene amplification reaction solution containing a plurality of primers binding to a plurality of polymorphic sites by sandwiching each site between primers,
a typing reagent holding part for holding a typing reagent prepared so as to correspond to a plurality of the polymorphic sites,
an amplification part for controlling the temperature of a reaction solution having the biological sample added to the gene amplification reaction solution to amplify genome DNA in the reaction solution,
a typing reaction part which has a probe fixing part for holding a probe emitting fluorescence corresponding to each of a plurality of the polymorphic sites and which controls the temperature of a reaction solution of the typing reagent and genome DNA amplified in the amplification part in order to react the reaction solution with each probe,
a dispensing device which can transfer to the sample arrangement part, the amplification reagent holding part, the typing reagent holding part, the amplification part and the typing reaction part and which dispenses a sample, an amplification reagent, a typing reagent, and a reaction solution of the sample and these reagents to predetermined positions,
a fluorescence detector for detecting fluorescence upon irradiation of each probe fixing part in the typing reaction part with an exciting light, and
a control part for controlling the temperatures of the amplification part and the typing reaction part and for controlling the dispensing operation of the dispensing device and the detection operation of the fluorescence detector.

7. The apparatus for detecting gene polymorphism according to claim 6, wherein the typing reaction part is provided in each of the probe fixing parts with a concave part, an upper part of which is opened to feed a reaction solution.

8. The apparatus for detecting gene polymorphism according to claim 7, which further comprises an oil holding part for holding an oil for preventing the evaporation of the reaction solution,
the dispensing device being capable of dispensing the oil to the concave portion before or after dispensing the reaction solution to the concave portion.

9. The apparatus for detecting gene polymorphism according to claim 6, wherein the typing reaction part is provided in each of the probe fixing parts with a flow path through which a reaction solution is fed.

10. The apparatus for detecting gene polymorphism according to claim 9, wherein the flow path is provided in each of the probe fixing parts with an inlet for feeding a reaction solution and an outlet for discharge.

11. The apparatus for detecting gene polymorphism according to claim 9, wherein the flow path is connected to a common inlet for feeding a reaction solution and a common outlet for discharge.

12. The apparatus for detecting gene polymorphism according to claim 9, wherein the probe fixing part is a partially broadened part in the flow path.

13. The apparatus for detecting gene polymorphism according to claim 6, wherein the typing reaction part is provided with a flow path having a plurality of the probe fixing parts formed therein.

14. The apparatus for detecting gene polymorphism according to claim 6, wherein the sample arrangement part and the amplification part share a temperature regulation part.

15. An examination reagent kit wherein an amplification reagent holding part in which a gene amplification reaction solution containing a plurality of primers binding to a plurality of polymorphic sites by sandwiching each site between primers has been held, a typing reagent holding part in which a typing reagent prepared so as to correspond to a plurality of the polymorphic sites has been held, and a plurality of probe fixing parts in which probes emitting fluorescence corresponding to each of a plurality of the polymorphic sites have been separately held, are integrally formed.

16. The examination reagent kit according to claim 15, wherein a diluent holding part in which a diluent for diluting a sample has been held is further integrally formed.

17. The examination reagent kit according to claim 15, wherein each of the holding parts is made of a soft material.

18. An apparatus for detecting gene polymorphism, comprising:
an examination reagent kit-mounting part for mounting the examination reagent kit according to claim 15,
an amplification part for controlling temperature of a reaction solution of the gene amplification reaction solution and a biological sample in order to amplify genome DNA in the reaction solution in the amplification reagent holding part,
a typing reaction part for controlling temperature of a reaction solution of the typing reagent and the genome DNA amplified in the amplification part in order to react the reaction solution with the probe in the probe fixing part,
a liquid transferring device for transferring liquid from the amplification reagent holding part to the typing reagent holding part and for transferring liquid from the typing reagent holding part to each of the probe fixing parts,
a fluorescence detector for detecting fluorescence upon irradiation of each of the probe fixing parts with an exciting light, and
a control part for controlling the temperatures of the amplification part and the typing reaction part, the liquid transferring operation of the liquid transferring device, and the detection operation of the fluorescence detector.

19. The apparatus for detecting gene polymorphism according to claim 18, wherein the liquid transferring device is a dispensing device equipped with a dispensing nozzle and arranged so as to be movable to a necessary place.

20. The apparatus for detecting gene polymorphism according to claim 18, wherein the examination reagent kit has each of the holding parts being made of a soft material, and
the liquid transferring device is a pressing device for pressing to deform each of the holding parts thereby transferring the liquid.

21. A diagnostic device comprising:
the apparatus for detecting gene polymorphism according to claim 6,
a database in which diagnostic values on a specific polymorphism or a combination of polymorphisms are memorized, and
a display unit for displaying diagnostic values by reading out from the database on the basis of the result of polymorphism detected by the apparatus for detecting gene polymorphism.

22. A diagnostic device comprising:
the apparatus for detecting gene polymorphism according to claim 18,
a database in which diagnostic values on a specific polymorphism or a combination of polymorphisms are memorized, and
a display unit for displaying diagnostic values by reading out from the database on the basis of the result of polymorphism detected by the apparatus for detecting gene polymorphism.

23. The method of detecting gene polymorphism according to claim 2, wherein the amplification step makes use of a PCR method.

24. The method of detecting gene polymorphism according to claim 2, wherein the typing step makes use of an invader method or a TaqMan PCR method.
